(19) United States
(12) Patent Application Publication
(10) Pub. No.: US 2026/0124467 A1
(43) Pub. Date: May 7, 2026

(54) ELECTRONIC PORTAL IMAGING DEVICE PREDICTION METHOD AND APPARATUS

(71) Applicant: Siemens Healthineers International AG, Steinhausen (CH)

(72) Inventor: Ari Harju, Espoo (FI)

(21) Appl. No.: 18/937,334

(22) Filed: Nov. 5, 2024

(51) Int. Cl. A61N 5/10

(52) U.S. Cl. CPC A61N 5/1039; A61N 5/1031; A61N 5/1038; A61N 2005/1054

(57) ABSTRACT

A control circuit may, before administering therapeutic radiation to a particular patient using a particular radiation treatment apparatus with an optimized radiation treatment plan, access an electronic portal imaging device prediction neural network that was trained with a plurality of training items each comprising information regarding a previous radiation treatment plan and corresponding electronic portal imaging device signal information and then input information regarding the optimized radiation treatment plan to the electronic portal imaging device prediction neural network and output a corresponding predicted electronic portal imaging device signal.

### TECHNICAL FIELD

These teachings relate generally to treating a patient's planning target volume with energy pursuant to an energy-based treatment plan.

### BACKGROUND

The use of energy to treat medical conditions comprises a known area of prior art endeavor. For example, radiation therapy comprises an important component of many treatment plans for reducing or eliminating unwanted tumors. Unfortunately, applied energy does not inherently discriminate between unwanted material and adjacent tissues, organs, or the like that are desired or even critical to continued survival of the patient. As a result, energy such as radiation is ordinarily applied in a carefully administered manner to at least attempt to restrict the energy to a given target volume. A so-called radiation treatment plan often serves in the foregoing regards.

A radiation treatment plan typically comprises specified values for each of a variety of treatment-apparatus parameters during each of a plurality of sequential fields. Treatment plans for radiation treatment sessions are often automatically generated through a so-called optimization process. As used herein, “optimization” will be understood to refer to improving a candidate treatment plan without necessarily ensuring that the optimized result is, in fact, the singular best solution. Such optimization often includes automatically adjusting one or more physical treatment parameters (often while observing one or more corresponding limits in these regards) and mathematically calculating a likely corresponding treatment result (such as a level of dosing) to identify a given set of treatment parameters that represent a good compromise between the desired therapeutic result and avoidance of undesired collateral effects.

Electronic portal imaging devices are digital imaging systems used in radiation therapy to verify the position and alignment of the therapeutic beam with respect to the patient's anatomy, typically in real-time. Electronic portal imaging devices are also sometimes used to support plan verification. In that case, there is nothing in the beamline by way of patient or phantom object. Mounted on the linear accelerator, the electronic portal imaging device captures high-resolution images of the treatment area by detecting the radiation that passes through the patient during treatment. These images can then be compared with reference images to help ensure that the radiation is being delivered accurately to the target while minimizing exposure to surrounding healthy tissue.

### BRIEF DESCRIPTION OF DRAWINGS

Various needs are at least partially met through provision of the described in the following detailed description, particularly when studied in conjunction with the drawings, wherein:

FIG. 1 comprises a block diagram as configured in accordance with various embodiments of these teachings;

FIG. 2 comprises a flow diagram as configured in accordance with various embodiments of these teachings; and

FIG. 3 comprises a flow diagram as configured in accordance with various embodiments of these teachings.

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present teachings. Also, common but well-understood elements that are useful or necessary in a commercially feasible embodiment are often not depicted in order to facilitate a less obstructed view of these various embodiments of the present teachings. The terms and expressions used herein have the ordinary technical meaning as is accorded to such terms and expressions by persons skilled in the technical field as set forth above except where different specific meanings have otherwise been set forth herein. The word “or” when used herein shall be interpreted as having a disjunctive construction rather than a conjunctive construction unless otherwise specifically indicated.

### DETAILED DESCRIPTION

Generally speaking, these various embodiments include actions taken before administering therapeutic radiation to a particular patient using a particular radiation treatment apparatus with an optimized radiation treatment plan. By one approach, a control circuit accesses an electronic portal imaging device prediction neural network that was trained with a plurality of training items each comprising information regarding a previous radiation treatment plan and corresponding electronic portal imaging device signal information and then inputs information regarding the optimized radiation treatment plan to the electronic portal imaging device prediction neural network which then outputs a corresponding predicted electronic portal imaging device signal.

By one approach, the aforementioned information regarding a previous radiation treatment plan can comprise physical parameter settings for the particular radiation treatment apparatus corresponding to previously-administered radiation treatment plans.

By one approach, the aforementioned information regarding the corresponding electronic portal imaging device signal information can comprise historical information corresponding to previously-administered radiation treatment plans and/or information calculated as a function of a corresponding previous radiation treatment plan.

These teachings are highly flexible in practice and will accommodate a variety of modifications and/or supplemental features. As one example, these teachings will accommodate facilitating a comparison of the corresponding predicted electronic portal imaging device signal with a reference electronic portal imaging device signal. That reference electronic portal imaging device signal can, for example, correspond to an electronic portal imaging device signal for the optimized radiation treatment plan. As another example, when the aforementioned comparison of the corresponding predicted electronic portal imaging device signal with the reference electronic portal imaging device signal is acceptable, these teachings will accommodate administering the therapeutic radiation to the particular patient using the particular radiation treatment apparatus with the optimized radiation treatment plan.

As noted above, these teachings will accommodate using a neural network. In those regards, these teachings will accommodate providing a training corpus comprising a plurality of training items that each comprise information regarding a radiation treatment plan and corresponding electronic portal imaging device signal information and then training a neural network using that training corpus to provide an electronic portal imaging device prediction neural network.

By one approach, the aforementioned plurality of training items that each comprise information regarding a radiation treatment plan can comprise a plurality of training items that each comprise information regarding a previously-administered radiation treatment plan. The aforementioned corresponding electronic portal imaging device signal information can comprise, for example, real-world signal information generated by a corresponding previously-administered radiation treatment plan and/or information generated with respect to a corresponding previously-administered radiation treatment plan.

By another approach, if desired, the aforementioned plurality of training items that each comprise information regarding a radiation treatment plan can comprise a plurality of training items that each comprise information regarding a not-previously-administered radiation treatment plan and/or the corresponding electronic portal imaging device signal information can comprise calculated signal information generated with respect to a corresponding not-previously-administered radiation treatment plan.

So configured, these teachings can facilitate predicting an electronic portal imaging device signal prior to administering the radiation to the patient with considerable accuracy. That accuracy, in turn (coupled with the speed of attaining that result), makes the resultant predicted signal useful for such purposes as quality assurance.

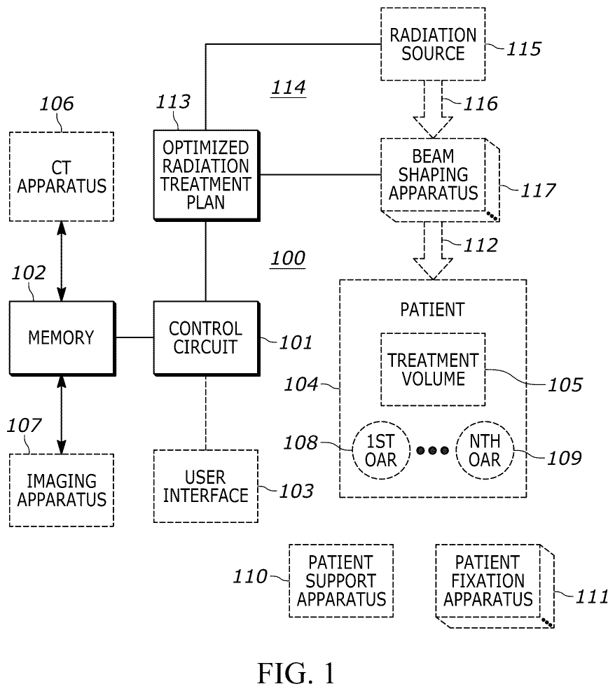

These and other benefits may become clearer upon making a thorough review and study of the following detailed description. Referring now to the drawings, and in particular to FIG. 1, an illustrative apparatus 100 that is compatible with many of these teachings will first be presented.

In this particular example, the enabling apparatus 100 includes a control circuit 101. Being a “circuit,” the control circuit 101 therefore comprises structure that includes at least one (and typically many) electrically-conductive paths (such as paths comprised of a conductive metal such as copper or silver) that convey electricity in an ordered manner, which path(s) will also typically include corresponding electrical components (both passive (such as resistors and capacitors) and active (such as any of a variety of semiconductor-based devices) as appropriate) to permit the circuit to effect the control aspect of these teachings.

Such a control circuit 101 can comprise a fixed-purpose hard-wired hardware apparatus (including but not limited to an application-specific integrated circuit (ASIC) (which is an integrated circuit that is customized by design for a particular use, rather than intended for general-purpose use), a field-programmable gate array (FPGA), and the like) or can comprise a partially or wholly-programmable hardware apparatus (including but not limited to microcontrollers, microprocessors, and the like). These architectural options for such structures are well known and understood in the art and require no further description here. This control circuit 101 is configured (for example, by using corresponding programming as will be well understood by those skilled in the art) to carry out one or more of the steps, actions, and/or functions described herein.

It will be appreciated that the control circuit 101 may comprise a single integrated apparatus or may comprise a plurality of such circuits that work in cooperation with one another.

The control circuit 101 operably couples to a memory 102. This memory 102 may be integral to the control circuit 101 or can be physically discrete (in whole or in part) from the control circuit 101 as desired. This memory 102 can also be local with respect to the control circuit 101 (where, for example, both share a common circuit board, chassis, power supply, and/or housing) or can be partially or wholly remote with respect to the control circuit 101 (where, for example, the memory 102 is physically located in another facility, metropolitan area, or even country as compared to the control circuit 101). As with the control circuit 101, the memory 102 may comprise a singular structure or may comprise a plurality of memory apparatuses that collectively comprise the “memory” of this apparatus 100.

In addition to information such as optimization information for a particular patient and information regarding a particular radiation treatment apparatus as described herein, this memory 102 can serve, for example, to non-transitorily store the computer instructions that, when executed by the control circuit 101, cause the control circuit 101 to behave as described herein. (As used herein, this reference to “non-transitorily” will be understood to refer to a non-ephemeral state for the stored contents (and hence excludes when the stored contents merely constitute signals or waves) rather than volatility of the storage media itself and hence includes both non-volatile memory (such as read-only memory (ROM) as well as volatile memory (such as a dynamic random access memory (DRAM).) In addition to the foregoing, the control circuit 101 can be configured as a neural network as described herein. Alternatively, the apparatus 100 can include a discrete neural network that serves as described below. As one non-limiting illustrative example, the neural network can comprise, at least in part, a convolutional neural network. A convolutional neural network consists of multiple layers that automatically and adaptively learn spatial hierarchies of features from input information (such as input images). These layers include convolutional layers that apply a number of filters to the input for feature detection, pooling layers that reduce the spatial size of the representation and the number of parameters, fully connected layers that perform classification based on the features detected by convolutional and pooling layers, and normalization layers that help with network training by normalizing the inputs to each layer. Each convolutional layer typically uses a small local receptive field to focus on local input patterns. Through backpropagation, the network adjusts its weights to minimize a loss function, improving its feature detection and classification abilities for the task at hand.

By one optional approach the control circuit 101 also operably couples to a user interface 103. This user interface 103 can comprise any of a variety of user-input mechanisms (such as, but not limited to, keyboards and keypads, cursor-control devices, touch-sensitive displays, speech-recognition interfaces, gesture-recognition interfaces, and so forth) and/or user-output mechanisms (such as, but not limited to, visual displays, audio transducers, printers, and so forth) to facilitate receiving information and/or instructions from a user and/or providing information to a user.

If desired the control circuit 101 can also operably couple to a network interface (not shown). So configured the control circuit 101 can communicate with other elements (both within the apparatus 100 and external thereto) via the network interface. Network interfaces, including both wireless and non-wireless apparatuses, are well understood in the art and require no particular elaboration here.

By one approach, a computed tomography apparatus 106 and/or other imaging apparatus 107 as are known in the art can source some or all of any desired patient-related imaging information.

In this illustrative example the control circuit 101 is configured to ultimately output an optimized energy-based treatment plan (such as, for example, an optimized radiation treatment plan 113). This energy-based treatment plan typically comprises specified values for each of a variety of treatment-apparatus parameters during each of a plurality of sequential exposure fields. In this case the energy-based treatment plan is generated through an optimization process, examples of which are provided further herein.

By one approach the control circuit 101 can operably couple to an energy-based treatment apparatus 114 that is configured to deliver therapeutic energy 112 to a corresponding patient 104 having at least one treatment volume 105 and also one or more organs-at-risk (represented in FIG. 1 by a first through an Nth organ-at-risk 108 and 109) in accordance with the optimized energy-based treatment plan 113. These teachings are generally applicable for use with any of a wide variety of energy-based treatment apparatus/apparatuses. In a typical application setting the energy-based treatment apparatus 114 will include an energy source such as a radiation source 115 of ionizing radiation 116.

By one approach this radiation source 115 can be selectively moved via a gantry along an arcuate pathway (where the pathway encompasses, at least to some extent, the patient themselves during administration of the treatment). The arcuate pathway may comprise a complete or nearly complete circle as desired. By one approach the control circuit 101 controls the movement of the radiation source 115 along that arcuate pathway, and may accordingly control when the radiation source 115 starts moving, stops moving, accelerates, de-accelerates, and/or a velocity at which the radiation source 115 travels along the arcuate pathway.

As one illustrative example, the radiation source 115 can comprise, for example, a radio-frequency (RF) linear particle accelerator-based (linac-based) x-ray source. A linac is a type of particle accelerator that greatly increases the kinetic energy of charged subatomic particles or ions by subjecting the charged particles to a series of oscillating electric potentials along a linear beamline, which can be used to generate ionizing radiation (e.g., X-rays) 116 and high energy electrons.

A typical energy-based treatment apparatus 114 may also include one or more support apparatuses 110 (such as a couch) to support the patient 104 during the treatment session, one or more patient fixation apparatuses 111, a gantry or other movable mechanism to permit selective movement of the radiation source 115, and one or more energy-shaping apparatuses (for example, beam-shaping apparatuses 117 such as jaws, multi-leaf collimators, and so forth) to provide selective energy shaping and/or energy modulation as desired.

In a typical application setting, it is presumed herein that the patient support apparatus 110 is selectively controllable to move in any direction (i.e., any X, Y, or Z direction) during an energy-based treatment session by the control circuit 101. As the foregoing elements and systems are well understood in the art, further elaboration in these regards is not provided here except where otherwise relevant to the description.

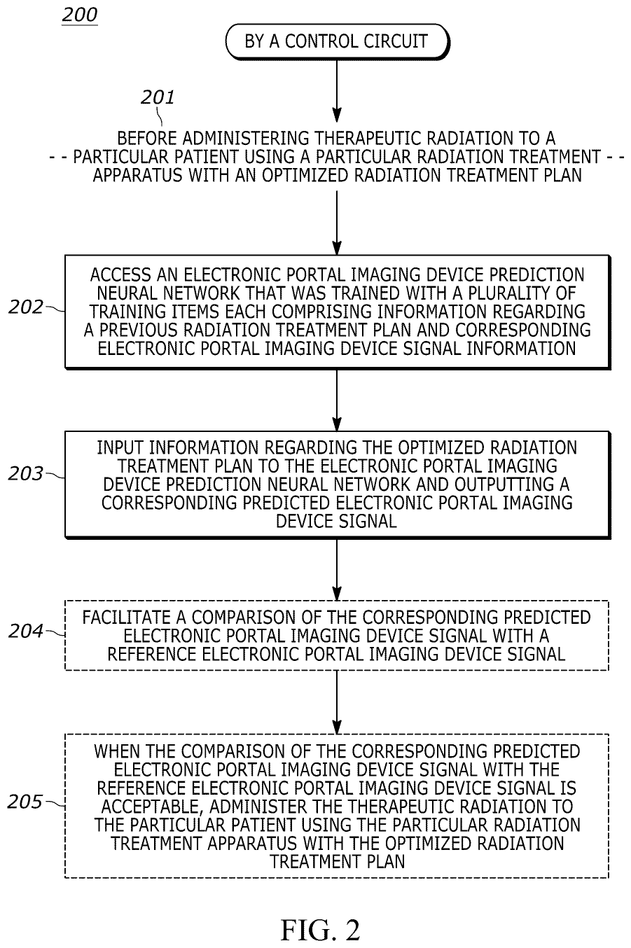

Referring now to FIG. 2, a process 200 that can be carried out, for example, in conjunction with the above-described application setting (and more particularly via the aforementioned control circuit 101) will be described. Generally speaking, this process 200 serves to facilitate outputting a predicted electronic portal imaging device signal that corresponds to a not-yet-administered optimized radiation treatment plan.

As denoted by reference numeral 201, this process 200 takes place before administering therapeutic radiation 112 to a particular patient 104 using a particular radiation treatment apparatus 114 with an optimized radiation treatment plan 113.

At block 202, this process 200 provides for accessing an electronic portal imaging device prediction neural network. This neural network, by one illustrative approach, was previously trained with a plurality of training items that each comprise information regarding a previous radiation treatment plan and corresponding electronic portal imaging device signal information.

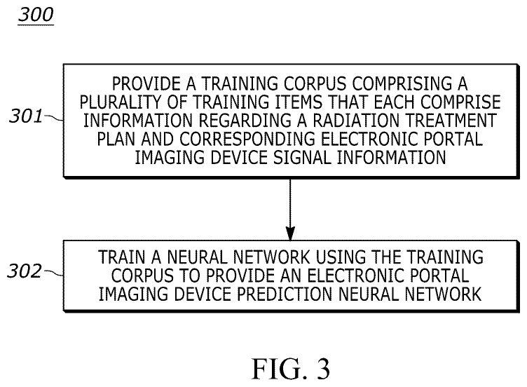

In the foregoing regards, and referring momentarily to FIG. 3, a training process 300 can include, at block 301, providing a training corpus comprising a plurality of training items that each comprise information regarding a radiation treatment plan and corresponding electronic portal imaging device signal information. The information regarding a radiation treatment plan can comprise image-based information, if desired. Examples include, but are not limited to, two-dimensional fluence maps, multi-leaf collimator apertures, and so forth.

As one illustrative example, the plurality of training items that each comprise information regarding a radiation treatment plan can comprise information regarding one or more previously-administered radiation treatment plans. In lieu of the foregoing, or in combination therewith, the corresponding electronic portal imaging device signal information can comprise real-world signal information previously generated by a corresponding previously-administered radiation treatment plan and/or may comprise calculated signal information generated with respect to a corresponding previously-administered radiation treatment plan.

In lieu of the foregoing, or in combination therewith, some or all of the plurality of training items that each comprise information regarding a radiation treatment plan may comprise a plurality of training items that each comprise information regarding a not-previously-administered radiation treatment plan. Similarly, if desired, the corresponding electronic portal imaging device signal information can comprise calculated signal information generated with respect to a corresponding not-previously-administered radiation treatment plan.

At block 302, this approach provides for training a neural network using the aforementioned training corpus to thereby provide an electronic portal imaging device prediction neural network.

Referring again to FIG. 2, at block 203 the control circuit 101 inputs information regarding the optimized radiation treatment plan 113 to the aforementioned electronic portal imaging device prediction neural network and outputs a corresponding predicted electronic portal imaging device signal. That output signal may, or may not, include one or more images as desired.

That predicted signal can be utilized and leveraged in a variety of ways. As one illustrative example, and referring to optional block 204, the control circuit 101 can facilitate a comparison of the corresponding predicted electronic portal imaging device signal with a reference electronic portal imaging device signal (such as, but not limited to, an electronic portal imaging device signal for the optimized radiation treatment plan 113).

By one approach, these teachings are carried out in an application setting that provides for consistent and locked beam information. Known beam characteristics can be very helpful when interpreting and/or comparing reference electronic portal image device signals.

By one approach, the foregoing comparison can comprise a comparison of the predicted image to a reference image. By one approach, that comparison can comprise a visual assessment by a trained technician/user facilitated, for example, via the aforementioned user interface 103. These teachings will also accommodate, if desired, a partial or wholly-automated comparison. These teachings are also flexible enough to accommodate a comparison of data types other than images.

At optional block 205 (and when, for example, this activity is part of a quality assurance process conducted prior to administering an optimized radiation treatment plan), when the aforementioned comparison of the corresponding predicted electronic portal imaging device signal with the reference electronic portal imaging device signal is acceptable, these teachings can provide for then administering the therapeutic radiation 112 to the particular patient 104 using the particular radiation treatment apparatus 114 with the optimized radiation treatment plan 113. Conversely, when the comparison yields an unacceptable result, that information can be, for example, communicated to the user to prompt a corresponding analysis and/or re-optimization of a radiation treatment plan.

The aforementioned acceptability can be based, for example, upon determining whether one or more comparison metrics are within a satisfactory range of differentiation. The latter may be represented, for example, by one or more corresponding thresholds.

Using the foregoing predicted results in a quality assurance context as described above, though likely surprising to persons of ordinarily skill in the art, is nevertheless possible because the resultant accuracy of such predictions (and the speed by which those results can be attained) is considerably better than what is presently being achieved by other prediction approaches. While the predicted signal is not dose per se, the predicted signal is a sufficiently close-enough approximation to serve as a useful surrogate for dose in quality assurance usage.

It will be appreciated that these teachings have additional applicability in other application settings. As but one illustrative example in those regards, these teachings can be leveraged to predict transit dosimetry images (transit dosimetry images being images taken while administering a radiation treatment session for the particular patient). Transit dosimetry images are typically captured by measuring the radiation that passes through the patient during treatment. This is typically accomplished using an electronic portal imaging device that is mounted on the linear accelerator that delivers the radiation. So configured, the electronic portal imaging device captures images of the radiation beam after it exits the patient, such images sometimes referred to as “exit dose” images. The predicted electronic portal imaging device signals provided by these teachings can serve to either supplement or to substitute for the foregoing practice.

Further aspects of these teachings are provided by the subject matter of the following clauses (where it will be understood that any of these clauses can be combined with any one of more of the other clauses as appropriate).
Clause 1. A method comprising: by a control circuit: before administering therapeutic radiation to a particular patient using a particular radiation treatment apparatus with an optimized radiation treatment plan: accessing an electronic portal imaging device prediction neural network that was trained with a plurality of training items each comprising information regarding a previous radiation treatment plan and corresponding electronic portal imaging device signal information; inputting information regarding the optimized radiation treatment plan to the electronic portal imaging device prediction neural network and outputting a corresponding predicted electronic portal imaging device signal.
Clause 2. The method of clause 1 wherein the information regarding a previous radiation treatment plan comprises physical parameter settings for the particular radiation treatment apparatus corresponding to previously-administered radiation treatment plans.
Clause 4. The method of either of clause 1 or 2 wherein the information regarding the corresponding electronic portal imaging device signal information comprises historical information corresponding to previously-administered radiation treatment plans.
Clause 5. The method of any of clause 1 through 4 wherein the information regarding the corresponding electronic portal imaging device signal information comprises information calculated as a function of a corresponding previous radiation treatment plan.
Clause 6. The method of any of clause 1 through 5 further comprising: facilitating a comparison of the corresponding predicted electronic portal imaging device signal with a reference electronic portal imaging device signal.
Clause 7. The method of clause 6 wherein the reference electronic portal imaging device signal corresponds to an electronic portal imaging device signal for the optimized radiation treatment plan.
Clause 8. The method of clause 6 further comprising: when the comparison of the corresponding predicted electronic portal imaging device signal with the reference electronic portal imaging device signal is acceptable, administering the therapeutic radiation to the particular patient using the particular radiation treatment apparatus with the optimized radiation treatment plan.
Clause 9. A method comprising: providing a training corpus comprising a plurality of training items that each comprise information regarding a radiation treatment plan and corresponding electronic portal imaging device signal information; training a neural network using the training corpus to provide an electronic portal imaging device prediction neural network.
Clause 10. The method of clause 9 wherein the plurality of training items that each comprise information regarding a radiation treatment plan comprises a plurality of training items that each comprise information regarding a previously-administered radiation treatment plan.
Clause 11. The method of clause 10 wherein the corresponding electronic portal imaging device signal information comprises real-world signal information generated by a corresponding previously-administered radiation treatment plan.
Clause 12. The method of clause 10 wherein the corresponding electronic portal imaging device signal information comprises calculated signal information generated with respect to a corresponding previously-administered radiation treatment plan.
Clause 13. The method of any of clause 9 through 12 wherein the plurality of training items that each comprise information regarding a radiation treatment plan comprises a plurality of training items that each comprise information regarding a not-previously-administered radiation treatment plan.
Clause 14. The method of clause 13 wherein the corresponding electronic portal imaging device signal information comprises calculated signal information generated with respect to a corresponding not-previously-administered radiation treatment plan.
Clause 15. An apparatus comprising: a control circuit configured to, before therapeutic radiation is administered to a particular patient using a particular radiation treatment apparatus with an optimized radiation treatment plan: access an electronic portal imaging device prediction neural network that was trained with a plurality of training items each comprising information regarding a previous radiation treatment plan and corresponding electronic portal imaging device signal information; input information regarding the optimized radiation treatment plan to the electronic portal imaging device prediction neural network and outputting a corresponding predicted electronic portal imaging device signal.
Clause 16. The apparatus of clause 15 wherein the information regarding a previous radiation treatment plan comprises physical parameter settings for the particular radiation treatment apparatus corresponding to previously-administered radiation treatment plans.
Clause 17. The apparatus of either clause 15 or 16 wherein the information regarding the corresponding electronic portal imaging device signal information comprises historical information corresponding to previously-administered radiation treatment plans.
Clause 18. The apparatus of any of clause 15 through 17 wherein the information regarding the corresponding electronic portal imaging device signal information comprises information calculated as a function of a corresponding previous radiation treatment plan.
Clause 19. The apparatus of any of clause 15 through 18 wherein the control circuit is further configured to: facilitate a comparison of the corresponding predicted electronic portal imaging device signal with a reference electronic portal imaging device signal.
Clause 20. The apparatus of clause 19 wherein the reference electronic portal imaging device signal corresponds to an electronic portal imaging device signal for the optimized radiation treatment plan.

Those skilled in the art will recognize that a wide variety of modifications, alterations, and combinations can be made with respect to the above-described embodiments without departing from the scope of the invention, and that such modifications, alterations, and combinations are to be viewed as being within the ambit of the inventive concept.

1. A method comprising:
by a control circuit:
before administering therapeutic radiation to a particular patient using a particular radiation treatment apparatus with an optimized radiation treatment plan:
accessing an electronic portal imaging device prediction neural network that was trained with a plurality of training items each comprising information regarding a previous radiation treatment plan and corresponding electronic portal imaging device signal information;
inputting information regarding the optimized radiation treatment plan to the electronic portal imaging device prediction neural network and outputting a corresponding predicted electronic portal imaging device signal.

2. The method of claim 1 wherein the information regarding a previous radiation treatment plan comprises physical parameter settings for the particular radiation treatment apparatus corresponding to previously-administered radiation treatment plans.

3. The method of claim 1 wherein the information regarding the corresponding electronic portal imaging device signal information comprises historical information corresponding to previously-administered radiation treatment plans.

4. The method of claim 1 wherein the information regarding the corresponding electronic portal imaging device signal information comprises information calculated as a function of a corresponding previous radiation treatment plan.

5. The method of claim 1 further comprising:
facilitating a comparison of the corresponding predicted electronic portal imaging device signal with a reference electronic portal imaging device signal.

6. The method of claim 5 wherein the reference electronic portal imaging device signal corresponds to an electronic portal imaging device signal for the optimized radiation treatment plan.

7. The method of claim 5 further comprising:
when the comparison of the corresponding predicted electronic portal imaging device signal with the reference electronic portal imaging device signal is acceptable, administering the therapeutic radiation to the particular patient using the particular radiation treatment apparatus with the optimized radiation treatment plan.

8. A method comprising:
providing a training corpus comprising a plurality of training items that each comprise information regarding a radiation treatment plan and corresponding electronic portal imaging device signal information;
training a neural network using the training corpus to provide an electronic portal imaging device prediction neural network.

9. The method of claim 8 wherein the plurality of training items that each comprise information regarding a radiation treatment plan comprises a plurality of training items that each comprise information regarding a previously-administered radiation treatment plan.

10. The method of claim 9 wherein the corresponding electronic portal imaging device signal information comprises real-world signal information generated by a corresponding previously-administered radiation treatment plan.

11. The method of claim 9 wherein the corresponding electronic portal imaging device signal information comprises calculated signal information generated with respect to a corresponding previously-administered radiation treatment plan.

12. The method of claim 8 wherein the plurality of training items that each comprise information regarding a radiation treatment plan comprises a plurality of training items that each comprise information regarding a not-previously-administered radiation treatment plan.

13. The method of claim 12 wherein the corresponding electronic portal imaging device signal information comprises calculated signal information generated with respect to a corresponding not-previously-administered radiation treatment plan.

14. An apparatus comprising:
a control circuit configured to, before therapeutic radiation is administered to a particular patient using a particular radiation treatment apparatus with an optimized radiation treatment plan:
access an electronic portal imaging device prediction neural network that was trained with a plurality of training items each comprising information regarding a previous radiation treatment plan and corresponding electronic portal imaging device signal information;
input information regarding the optimized radiation treatment plan to the electronic portal imaging device prediction neural network and outputting a corresponding predicted electronic portal imaging device signal.

15. The apparatus of claim 14 wherein the information regarding a previous radiation treatment plan comprises physical parameter settings for the particular radiation treatment apparatus corresponding to previously-administered radiation treatment plans.

16. The apparatus of claim 14 wherein the information regarding the corresponding electronic portal imaging device signal information comprises historical information corresponding to previously-administered radiation treatment plans.

17. The apparatus of claim 14 wherein the information regarding the corresponding electronic portal imaging device signal information comprises information calculated as a function of a corresponding previous radiation treatment plan.

18. The apparatus of claim 14 wherein the control circuit is further configured to:
facilitate a comparison of the corresponding predicted electronic portal imaging device signal with a reference electronic portal imaging device signal.

19. The apparatus of claim 18 wherein the reference electronic portal imaging device signal corresponds to an electronic portal imaging device signal for the optimized radiation treatment plan.
